(19) United States
(12) Patent Application Publication
(10) Pub. No.: US 2018/0104240 A1
(43) Pub. Date: Apr. 19, 2018

(54) AMELIORATION OF RADIATION-INDUCED COGNITIVE DYSFUNCTION

(71) Applicant: NEURALSTEM, INC., Germantown, MD (US)

(72) Inventor: Karl K. JOHE, Sunny Isles, FL (US)

(73) Assignee: NEURALSTEM, INC., Germantown, MD (US)

(21) Appl. No.: 15/787,590

(22) Filed: Oct. 18, 2017

Related U.S. Application Data
(60) Provisional application No. 62/409,789, filed on Oct. 18, 2016.

(51) Int. Cl. A61K 31/496; A61K 31/444; A61K 31/465; A61K 9/00

(52) U.S. Cl. CPC A61K 31/496; A61K 9/0053; A61K 31/465; A61K 31/444

(57) ABSTRACT

Treatment with 2-amino substituted nicotinamides ameliorates the cognitive dysfunction resulting from radiation of the brain.

### CROSS REFERENCE TO RELATED APPLICATIONS

This application claims priority from U.S. provisional application 62/409,789 filed 18 Oct. 2016. The contents of this document are incorporated by reference herein in their entirety.

### TECHNICAL FIELD

The invention relates to treatment of subjects who have undergone brain radiation treatment with compounds that ameliorate the cognitive deficits associated with this therapy. More particularly, it concerns the use of 2-amino substituted nicotinamides for this purpose.

### BACKGROUND ART

Malignancies of the brain are difficult to treat post-surgically due to the difficulty for pharmaceuticals to transit the blood-brain barrier and also to the sensitive nature of the treatment site. Radiation therapy has been used to improve progression-free survival. Although focused delivery has helped limit tissue injury such as radionecrosis and/or edema, it is inevitable that cognitive dysfunction occurs (cites). These deficits in cognitive function are experienced even if there is no morphological evidence of injury in the brain. These deficits include problems with learning and memory as well as problems with attention, concentration and executive functions such as planning and multitasking (cite).

Attempts have been made to employ antioxidant and anti-inflammatory therapies following radiation, but it does not appear that these are successful in overcoming the cognitive deficits caused by irradiation (cites).

These deficits may occur by virtue of depletion of neural stem and progenitor cells which reside in the neurogenic subgranular zone (SGZ) of the hippocampal dentate gyrus (DG). In addition, oxidative stress and inflammation further compromise the neurogenic niche (cites). It is therefore thought that stimulation of neurogenesis may be helpful in overcoming these problems.

A family of U.S. granted patents, represented by, for example, U.S. Pat. No. 8,362,262, discloses low molecular weight compounds that are capable of stimulating neuronal growth, and are able to permeate the blood-brain barrier (BBB). Subsequently, it was found that certain 2-amino-substituted nicotinamides were useful in treating depression, in particular, major depressive disorder in humans as described in PCT publication WO2015/195567, and corresponding U.S. Pat. No. 9,572,807. Oral administration of these compounds in such treatments is disclosed. However, nothing in these documents suggests that deficits in cognitive function caused by irradiation can be restored by these compounds despite their known ability to stimulate neuronal growth, and their ability to improve self-perceived cognition in depressed patients.

### DISCLOSURE OF THE INVENTION

It has now been found that certain 2-amino-substituted nicotinamides are especially useful in prevention or repair of various aspects of cognitive impairments that are sequelae of radiation treatment of the brain, or indeed the result of accidental exposure to radiation.

Accordingly, in one aspect, the invention is directed to a method to ameliorate the cognitive deficiencies associated with radiation of the brain by administering to a subject in need of such amelioration, a pharmaceutical composition wherein the active ingredient is a 2-amino-substituted nicotinamide or a pharmaceutically acceptable salt thereof. In particular, the 2-amino-substituted nicotinamide is of the formula:

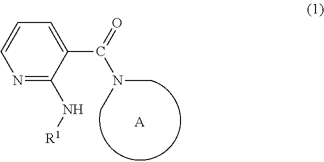

wherein R¹ is an alkyl of 3-8C and ring A is a 5- or 6-membered saturated ring optionally including an additional nitrogen which is unsubstituted or substituted with an additional nitrogen-containing substituent or a ring-opened form thereof.

Particular exemplified compounds include those of formula (2)

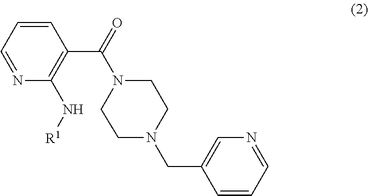

or formula (3)

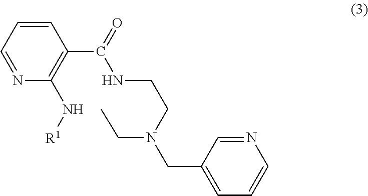

wherein R¹ is a branched alkyl group of 3-5C or of formula (4)

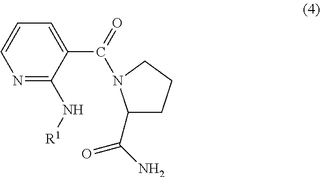

wherein R¹ is an alkyl group comprising a 5- or 6-membered ring.

In one embodiment, the compound of formula (2) is employed as a pharmaceutically acceptable salt, in particular the phosphate salt, and also, in particular, wherein R¹ is isoamyl.

### MODES OF CARRYING OUT THE INVENTION

The methods of the invention are directed to ameliorating the cognitive deficiencies associated with radiation treatment of the brain.

The active agents useful in the method of the invention have the general formula (1) noted above wherein R¹ is an alkyl of 3-8C and ring A is a 5- or 6-membered saturated ring optionally including an additional nitrogen or a ring-opened form thereof. Thus, R¹ may be, in formula (1), a straight or branched chain alkyl group of at least 3C, such as isopropyl, secondary butyl, n-butyl, isoamyl, sec-amyl, hexyl, isohexyl and the like or comprise a saturated ring. Preferably in formula (2) or (3), R¹ is a branched alkyl of 3-5C and, in formula (4), R¹ comprises a 5- or 6-membered saturated ring. Preferred embodiments of ring A are a piperidine or piperazine ring or ring opened forms thereof or a pyrrolidine ring.

Ring A may also be substituted with at least an additional nitrogen-containing substituent, including a substituent that comprises an additional pyridine ring such as pyridyl methyl, or pyridyl ethyl or is a simpler substituent such as a carboxamide. Preferred forms of ring A are shown in formulas (2), (3) and (4) above along with appropriate substituents.

The compounds of the invention are typically, but not always, administered in the form of their pharmaceutically acceptable salts such as halides, maleates, succinates, nitrates and the like. Particularly favored are phosphate salts.

The compounds of the invention are formulated in standard pharmaceutical formulations such as those found in Remington's Pharmaceutical Sciences, latest edition, Mack Publishing Co., Easton, Pa. and include formulations for oral administration and parenteral administration. Typically, the compounds are administered orally in the form of tablets, capsules or in formulations that are administered as syrups or any other standard formulation. In some instances, the formulations may be designed for delayed release or may be designed for more instantaneous delivery. A variety of formulations that would be suitable for the compounds of the invention is known in the art and is subject to the decision of the practitioner with regard to route of administration.

Dosage levels also depend on the judgment of the practitioner, but are generally in the range of 0.01 mg/kg to 1-2 g/kg.

In general, the subjects of the treatment will be humans, although it is useful to employ laboratory animals as well in order to assess appropriate dosages, routes of administration and formulations. Thus, the subjects of the invention include not only humans, but laboratory research animals such as rabbits, rats, mice and the like. In some instances, other mammalian subjects may be appropriate such as in veterinary contexts where the subject may be ovine, bovine or equine or the subject may be a companion animal such as dog or cat.

In some embodiments, the method of treatment further includes a concomitant diagnostic procedure whereby the effect of the treatment is evaluated at various timepoints during administration and/or after administration of the compositions of the invention. These evaluations include evaluation of, for example, novel place recognition, novel object recognition, object and place recognition and recognition of temporal order. The analyses may also include fear conditioning.

A particularly useful diagnostic is measurement by CogScreen, a computer-administered cognitive test battery required by the U.S. Federal Aviation Administration (FAA) for evaluation of the neurocognitive functioning of pilots and which has also played a key role in the FDA drug approval and labeling process (CogScreen LLC, St Petersburg, Fla.). This includes analysis of Shifting Attention Test-Arrow Color Accuracy a measure of executive functioning; Shifting Attention Test-Arrow Direction Reaction Time Correct, a measure of attention; Symbol Digit Coding-Delayed Recall Accuracy, a measure of memory and Shifting Attention Test-Instruction Number Incorrect, which is a measure of working memory. One or a combination of these aspects or a subset thereof may be employed.

The frequency of administration and dosage schedules is also dependent on the practitioner and the dose may be chronic and on a daily basis, weekly basis or more frequent, or a single dosage may suffice. In typical protocols, the compositions of the invention are administered orally daily beginning shortly before or after radiation and for 2 weeks to 2 months. The compounds of the invention may also be administered in combination with other active agents either in the same composition or sequentially.

The following examples illustrate, but do not limit the invention,

### Example 1

### Therapy and Evaluation of Cognitive Deficits

Ten (10) week old Long Evans (LE) rats (Crl:LE, Strain Code: 006; Charles River) were divided randomly into three groups (n=16/group):

Controls (Con) received oral gavage (vehicle only) and sham radiation.

Radiation recipients with no drug (IRR) administered oral gavage (vehicle only) and 27 Gy head-only fractionated radiation.

Radiation recipients having administered drug (IRR+NSI-189) received the compound of formula (2) wherein R¹=amyl as the phosphate salt by oral gavage. Drug was administered at 30 mg/kg (amount based exclusive of phosphate) and 27 Gy head-only fractionated radiation administered as in the IRR group. Rats in this group were administered NSI-189 in sterile vehicle (normal saline) as a 1× solution (15 mg/ml) starting 24 hours after the final dose of radiation and daily thereafter for 30 days. Sterile gavage needles with protected tips were used and the drug administered for less than a minute.

For IRR and IRR+NSI-189, rats were anesthetized [2.5% (vol/vol) isoflurane/oxygen], placed ventrally on the treatment table (X-RAD 320 irradiator; Precision X-Ray) without restraint, and positioned under a collimated (1.0 cm² diameter) beam for head-only radiation delivered at a dose rate of 1.0 Gy/min. Fractionation of 27 Gy was delivered over 3 separate doses of 8.67 Gy that were administered 48 hours apart.

Rats were weighed weekly to adjust all dosing for that week. During the last 3 days of oral gavage (4 weeks prior to sacrifice), animals were injected 3 times daily with BrdU (50 mg/kg) in order to enable testing of neuronal growth.

Rats were subjected to cognitive testing using spontaneous exploration tasks outlined below 1 week after termination of oral gavage (5 weeks post irradiation) over the course of 3 weeks. This was followed by cued fear conditioning. All data were collected using Noldus Ethovision v8.5 and later hand scored by a technician blind to the treatment groups. All spontaneous exploration test data are presented in exploration ratio format [(tₙₒᵥₑₗ/tₙₒᵥₑₗ+t_{familiar})].

Novel Place Recognition (NPR):

Rats were exposed to two identical objects in specific spatial locations within a test arena. One of the objects was moved to a new location and 1 hour later the rats were exposed to the relocated object. Rats that remember the previous spatial arrangement will spend more time exploring the object that has been moved to the new location. The results are shown in Table 1 as exploration ratios—i.e., time spent exploring the new location divided by the initial exploration time. Successful performance of the task has been shown to rely on intact hippocampal function (Morimoto, T., et al., Cell Transplant (2011) 20:1049-1064; Hara, K., et al., J. Neurosci. Res. (2007) 85:1240-1251).

TABLE 1
Exploration Ratio Means and 95% Confidence Intervals (CI)

| | | |
|---|---|---|
| Con. | mean = 63.3 | CI = 57.09-69.51 |
| IRR | mean = 45.93 | CI = 35.39-56.82 |
| IRR + NSI-189 | mean = 60.69 | CI = 52.96-68.41 |

A significant overall group effect was found (F_{(2,44)}=5.357; P=0.0083) for the exploration ratio to differ between groups. IRR animals spent significantly less time exploring the novel place compared to Con (P<0.05) and IRR+NSI-189 (P<0.05). IRR+NSI-189 did not differ significantly from Con.

Novel Object Recognition (NOR):

NOR task measures the exploration times for familiar objects and novel objects. Rats explored familiar and novel objects during this task. The results are shown in Table 2 as exploration ratios defined as time spent exploring familiar objects divided by time exploring novel objects.

TABLE 2
Exploration Ratio Means and 95% CI

| | | |
|---|---|---|
| Con. | mean = 68.30 | CI = 61.94, 74.66) |
| IRR | mean = 55.14 | CI = 48.18, 62.09 |
| IRR + NSI-189 | mean = 71.28 | CI = 63.50, 79.06 |

A significant overall group effect was found (F_{(2,43)}=6.37; P=0.0038) for the exploration ratios to differ between groups. Following a 5 min retention interval between familiarization and test phases, IRR animals spent significantly less time exploring the novel object compared to Con (P<0.05) and IRR+NSI-189 (P<0.01) groups, and Con and IRR+NSI-189 groups were statistically indistinguishable.

NOR tests the functional connectivity of the hippocampus and the medial prefrontal cortex (mPFC).

Object in Place (OiP):

Functionally intact rats exhibit a preference towards objects that have been moved to a novel location as shown by exploration ratios in Table 3. The exploration ratio is the ratio of the time exploring the object at the new location to the time spent at the old location.

TABLE 3
Exploration Ratio Means and 95% CI

| | | |
|---|---|---|
| Con | mean = 64.75 | CI = 59.98, 69.52 |
| IRR | mean = 54.85 | CI = 49.47, 60.23) |
| IRR + NSI-189 | mean = 64.60 | CI = 59.17, 70.02 |

Overall group effect for exploration ratios between the three groups was again found to differ significantly (F_{(2,43)}=5.146; P<0.01). IRR animals spent significantly less time exploring the novel placement of objects compared to Con (P<0.05) and IRR+NSI-189 (P<0.05), while Con and IRR+NSI-189 treated cohorts showed no significant differences. OiP is dependent on intact hippocampal, prefrontal and perirhinal cortical functions.

Temporal Order (TO):

In TO, rats were familiarized with two sets of objects, 3 hours apart, and after an additional 1 hour, were tested for exploration times using one of the same objects presented at each earlier time. The exploration ratio is the ratio of the time spent at the later time to that at the earlier time. Rats with intact hippocampal function show a preference for the first rather than the more recent object explored. The results are in Table 4. Total exploration of the objects during the familiarization and test phases were not found to differ statistically.

TABLE 4
Exploration Ratio Means and 95% CI

| | | |
|---|---|---|
| Con | mean = 64.68 | CI = 59.71, 69.64 |
| IRR | mean = 46.44 | CI = 36.55, 56.33 |
| IRR + NSI-189 | mean = 67.95 | CI = 61.15, 74.75 |

Analysis of recency discrimination in the TO task revealed an overall group effect [F_{(2,44)}=10.63; P=0.0002] and show that radiation exposure impaired recency memory as reflected by a reduced preference for the object presented prior to the last. Con and IRR+NSI-189 did not differ statistically.

Morphometric and Synaptic Analyses:

At the end of behavior assessment, animals (n=4/group) were given a barbiturate overdose and perfused using a saline/heparin solution and were then subjected to Golgi-Cox impregnation and staining of neurons as per the manufacturer's instructions (SuperGolgi kit, Bioenno Tech.), sectioned at 150 μm and counterstained with nuclear fast red. Morphometric analysis and total spine count of mature neurons in the hippocampus DG was carried out as described in detail previously (cite). The remaining animals were injected with BrdU for 3 days to measure neurogenesis prior to sacrifice.

Neurogenesis Results:

Radiation induced a significant (P<0.05) reduction in the number of proliferating (BrdU⁺) cells present in the hippocampal dentate gyrus, which was ameliorated by NSI-189 treatment, as the number of proliferating cells was returned to pre-irradiation levels.

To confirm the identity of the BrdU cells, dual immunohistochemical labeling was performed. Cells doubly-labeled for BrdU and NeuN provide for the quantification of mature neurons, and irradiation reduced significantly, the yield of BrdU⁺/NeuN⁺ cells in the hippocampal dentate gyrus. NSI-189 treatment returned the yield of BrdU⁺/NeuN⁺ cells to pre-irradiation levels.

Assessment of Hippocampal Volume

Unbiased stereology was used to assess potential differences in hippocampal between all cohorts.

Assessment of Neurogenesis

Remaining animals that were not used for Golgi-Cox staining (n=12/group) were given a barbiturate overdose and were perfused/fixated using 4% paraformaldehyde and tissue removed. Tissue was analyzed by immunohistochemistry.

1. A method to ameliorate cognitive deficiency associated with radiation treatment, which method comprises administering to a subject in need of such amelioration an effective amount of a 2-amino substituted nicotinamide or pharmaceutically acceptable salt thereof.

2. The method of claim 1 wherein the cognitive deficiency is a decrease in novel place recognition (NPR), a decrease in novel object recognition (NOR), a decrease in object in place (OiP) preference, a decrease in recognition of temporal order (TO) and/or an enhanced reaction to fear.

3. The method of claim 1 wherein the cognitive deficiency is a decrease in executive function and/or a decrease in attention and/or a decrease in memory and/or a decrease in working memory.

4. The method of claim 1 wherein the 2-amino substituted nicotinamide is of the formula: 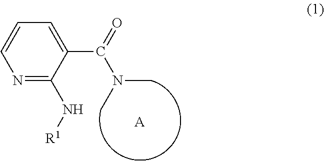
wherein R¹ is an alkyl of 3-8C and ring A is a 5- or 6-membered saturated ring optionally including an additional nitrogen which is unsubstituted or substituted with an additional nitrogen-containing substituent or a ring-opened form thereof.

5. The method of claim 4, wherein the 2-amino substituted nicotinamide is 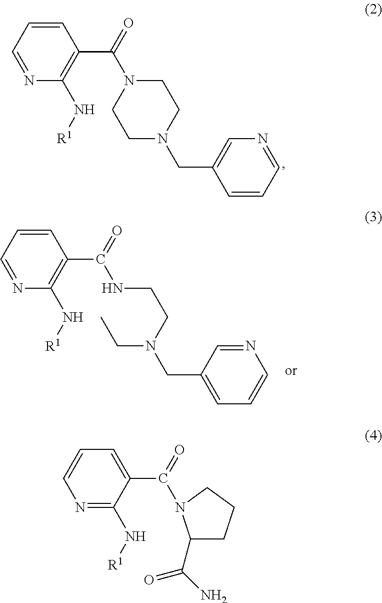
wherein R¹ is a branched alkyl group of 3-5C in formula (2) or (3) and is an alkyl group comprising a 5-6 membered saturated ring in formula (4).

6. The method of claim 5 wherein the 2-amino substituted nicotinamide is of formula (2) and R¹ is isoamyl.

7. The method of claim 1 wherein the 2-amino substituted nicotinamide is in the form a phosphate salt.

8. The method of claim 1 wherein said administering is oral.

9. The method of claim 1 wherein said administering is over a period of at least 6 weeks subsequent to the irradiation.

10. The method of claim 9 wherein said administering is over a period of at least 12 weeks subsequent to the irradiation.

11. The method of claim 1 wherein said administering is initiated within 6 hours after said irradiation.

12. The method of claim 1 which further includes subsequent testing of said subject for enhancement of recovery of said cognitive deficiency.

13. The method of claim 12 wherein said testing comprises novel place recognition (NPR) and/or novel object recognition (NOR) and/or object in place (OiP) and/or temporal order (TO).

14. The method of claim 12 wherein said testing comprises assessing executive function and/or attention and/or memory, and/or working memory.

15. The method of claim 7 wherein said administering is oral.

16. The phosphate salt of claim 7 wherein said administering is over a period of at least 6 weeks subsequent to the irradiation.

17. The method of claim 16 wherein said administering is over a period of at least 12 weeks subsequent to the irradiation.

18. The method of claim 7 wherein said administering is initiated within 6 hours after said irradiation.

19. The method of claim 7 which further includes subsequent testing of said subject for enhancement of recovery of said cognitive deficiency.

20. The method of claim 19 wherein said testing comprises novel place recognition (NPR) and/or novel object recognition (NOR) and/or object in place (OiP) and/or temporal order (TO).

21. The method of claim 19 wherein said testing comprises assessing executive function and/or attention and/or memory, and/or working memory.
